(19) United States
(12) Patent Application Publication
(10) Pub. No.: US 2024/0115351 A1
(43) Pub. Date: Apr. 11, 2024

(54) Alveolar Ridge Splitting Technique for Predictable Horizontat Ridge Augmentation

(71) Applicant: Sang-choon Cho, New York, NY (US)

(72) Inventor: Sang-choon Cho, New York, NY (US)

(21) Appl. No.: 18/272,186

(22) PCT Filed: Nov. 15, 2021
(86) PCT No.: PCT/US21/59343

§ 371 Date: Jul. 13, 2023

Related U.S. Application Data
(60) Provisional application No. 63/112,859, filed on Nov. 12, 2020.

(51) Int. Cl. A61C 1/08; A61C 8/00

(52) U.S. Cl. CPC A61C 1/084; A61C 8/0089

(57) ABSTRACT

Implant supported restorations have proven to be a predictable option for replacing missing teeth. In cases of inadequate quantity of bone, the bone volume can be increased by bone augmentation procedures. Several factors can affect bone regeneration, one of those is the morphology of the defect at the implant site. A defect surrounded by bony walls (an intra-osseous defect) is known to yield a highly successful regeneration. The purpose of this retrospective case series study was to present a new step-by-step surgical procedure known as the Custom Alveolar Ridge Splitting (CARS) technique for maxillary anterior ridge augmentation. This technique creates an intra-osseous defect while splitting and augmenting an atrophic ridge. Sixteen consecutive cases were treated with the CARS procedure. All cases were effectively treated with successful implant placement. All implants were restored and followed for twelve to twenty-four months after loading. According to this retrospective study, the CARS procedure is simple, successful, and predictable, and may be used as a surgical option for horizontal alveolar ridge augmentation in the anterior maxilla. A highly useful trephine bur design is also provided by this invention, especially useful for forming a green fracture to form walls to hold and support the bone graft material.

### INTRODUCTION TO AND BACKGROUND OF THE INVENTION

Implant-supported restorations have been proven to be a predictable option for successfully replacing missing teeth. However, problems have arisen when there was an insufficient volume of bone present in the edentulous alveolar ridge; generally, the minimum amount of bone required, to successfully support a permanent implant, is generally 2 mm of bone on the facial and oral aspects of the permanent implant. For the anterior maxilla, the goal of this therapy is to restore esthetics as well as function, which can present a challenge when the edentulous alveolar ridge is deficient in quantity and quality of bone. Alveolar bone loss; including contour changes, can occur by bone resorption and remodeling after tooth extraction, or may occur pathologically prior to tooth loss or extraction because of periodontal disease, periapical pathology, or trauma to teeth and bones. However, it has previously been found that in cases of inadequate quantity of bone, the bone volume can be increased by bone augmentation procedures in conjunction with or followed by implant placement. This was, however, not always successful.

### SUMMARY OF THE DISCLOSURE

To achieve esthetic and functionally stable implant-supported fixed prosthesis, a combination of soft and hard tissue augmentation procedures are often necessary. Despite advancements in bone regeneration techniques the outcomes in many cases were not highly predictable. Several factors can affect bone regeneration. One of those is the morphology of the defect at the implant site, which has been reported to be a critical factor for the success of bone augmentation. One type of defect, i.e., a deficiency of bone volume, surrounded by bony walls, is an intra-osseous defect, and this type of defect is known to yield a highly successful regeneration due to good blood and osteoblast supply and being well contained. However, the problems remain for those patients who suffer from an extra-osseous defect, with less bony walls, as the treatment has been found to be less predictable for bone augmentation procedures, and has been a continuing problem for dental surgeons and their patients.

The present invention resolves to a great extent the problems arising from an extra-osseous defect. In accordance with this invention, a new step-by-step surgical procedure, dubbed the Custom Alveolar Ridge Splitting (CARS) technique for maxillary anterior ridge augmentation, is available to greatly improve the likelihood of success in achieving a permanent implant replacement for lost teeth. The effectiveness of this procedure have been shown by the examples of patients who were treated in accordance with this new technique.

The text and drawings of U.S. Provisional application 63/112,859, filed Nov. 12, 2020 are hereby incorporated by reference as if fully repeated and included literally herein.

### A BRIEF DESCRIPTION OF THE DRAWINGS EXEMPLIFYING THE PRESENT INVENTIONS

FIG. 1 is a photograph showing a trephine bur drilling into the ridge bone where a tooth is missing;

FIG. 2 is a radiographic picture showing a completed implant in place;

FIG. 3 shows a 3-D printed model, of the maxillary anterior arch ridge area of an upper jaw, printed using a black plastic material, depicting the position of the missing tooth and the surrounding teeth with a guide cylinder in place in a space initially drilled as a pilot hole into the ridge bone;

FIGS. 4 and 4A are pictures of an elevation view and a plan view of a 3-D printed model as in FIG. 3, showing the final drilling by a trephine bur (FIG. 4), and the resultant green fracture is depicted in FIG. 4A (indicated by the arrow); and

FIGS. 5 and 5A are drawings depicting an elevation view and a plan view from the drilling end of the new trephine bur design preferred for effecting a green fracture in the dental ridge to create an extra-osseous defect.

### DETAILS OF THE PREFERRED FEATURES OF THE INVENTION

Sixteen consecutive cases were selected from patients who desired dental implants with a fixed prosthesis to replace their missing teeth in the anterior maxillary arch and had implants placed with the CARS procedure. All 16 cases were effectively treated with successful implant placement. Follow-up times were recorded for each of the implants placed. The CARS procedure follows a specific set of steps, but it can be modified according to the surgical scenario.

The preferred process of this invention is generally described as follows.

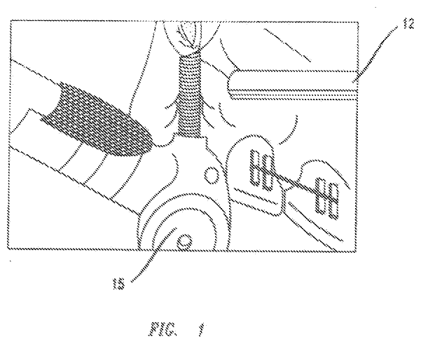

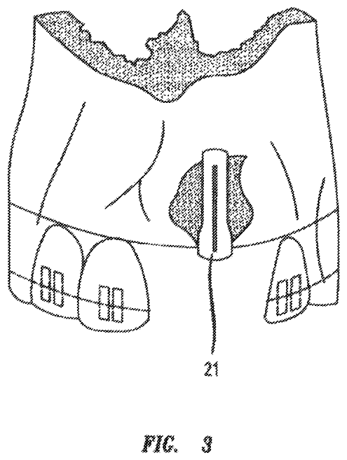

Following a CBCT of the surgical site, the point of entry of the trephine guide and trephine are determined on an axial section of the site. After elevation of a full thickness tissue flap, to expose the bone, i.e., the vertically facing ridge bone beneath the gum, the initial drilling is made into the ridge bone face with the help of a guide (FIG. 1), and a guide cylinder is placed into this first osteotomy, which was prosthetically selected for future implant placement (FIG. 3). A circular vertical cut is then created by an appropriately sized trephine bur (with the bur diameter similar to the diameter of the future implant) and guided by the guide cylinder (FIG. 3). The guide cylinder is then removed, and the final cut is made with the same trephine bur to the planned length (2 mm more than the future implant length).

During cutting, the surgeon evaluates the stability of the split segment. If the segment is stable, the second stage can be performed in the same surgery. If it is not stable, the flap is sutured, and reentry is performed 3 to 4 weeks later.

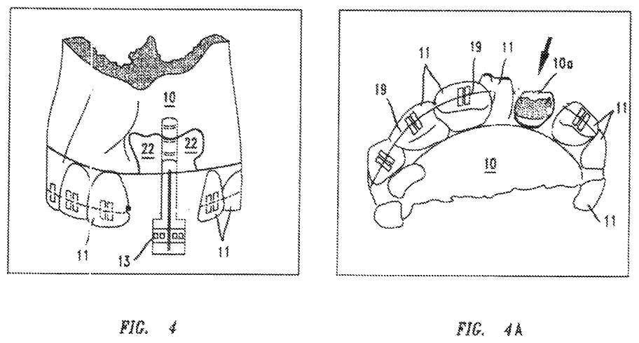

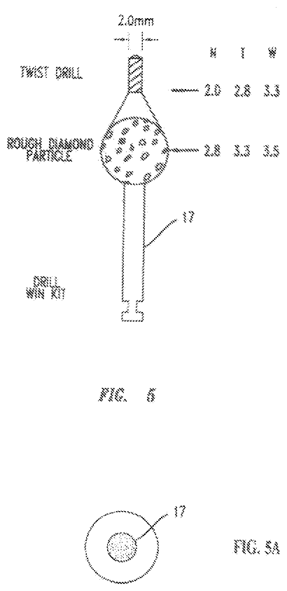

At the second stage, a greenstick fracture is created (as shown in FIG. 4A) by the trephine bur (or a small periosteal elevator or small bone carrier), although preferably a special trephine bur, shown in FIG. 5-SA and described below is used to gradually separate the sides of the drilled hole spreading the walls so that they can hold the graft material. The segment is moved buccally and wedged in the surrounding buccal bone plate. Again, the stability of the segment is evaluated. If good stability is achieved, implant placement can then be attempted. If the necessary stability is not found, bone grafting should be performed, to maintain the space, and the flap is sutured. The patient then returns 3 to 4 weeks later, and the last stage is performed, including osteotomy and implant placement. Tapered implants are the most indicated for this technique.

In the present study, implants were loaded 6 to 21 months after implant placement. In 11 of the tested cases, the CARS procedure was performed 3 to 4 weeks before implant placement. In 3 cases, the CARS procedure was performed simultaneously with implant placement and guided bone regeneration (GBR).

In the first case, the CARS procedure was performed 3 months prior to implant placement. In another case, the segment was fractured, and successful retreatment was performed 2 months later.

As part of the training for the surgery, as well as its planning, the technique for all cases included in this study was first performed on a 3D model of the patient's jaw (as shown in FIGS. 3, 4 and 4A), printed from the CBCT scan file. Using these models for surgical simulation familiarized the surgeon with the actual site and procedure that was to be performed on the patient. It also allowed the clinicians to experience the risks and helped them evaluate whether the site was more amenable to a two- or three-stage approach and whether the site required augmentation by a GBR procedure or any other procedure to manage any associated conditions.

### Examples of the Present Invention

The following two case reports are examples to illustrate the technique with its various aspects and procedures.

Case 1

A 22-year-old woman presented missing her maxillary right canine. She had a high smile line, 18 malocclusion, and parafunctional habits. The patient was first treated orthodontically to manage the malocclusion and parafunctional

habits before she was referred to restore her missing tooth. For this patient, the CARS technique was performed 4 weeks prior to implant placement. All procedures were performed under local anesthesia (2% lidocaine, 1:100,000; Henry Schein).

The initial surgery was performed with a crestal incision made at the edentulous site, extending from the maxillary right lateral incisor to the maxillary right first premolar, with intrasulcular incisions around the buccal aspects of the maxillary right lateral incisor and right first premolar.

This was followed by two vertical labial releasing incisions at the mesial aspect of the right lateral incisor and distal aspect of the right first premolar. A full-thickness flap was then elevated. Initial drilling was performed, and a guide cylinder was placed in the area that had been prosthetically selected for a future implant. A circular vertical cut was created with a 4.3-mm-diameter trephine bur (Straumann) guided by the guide cylinder. The guide cylinder was then removed, and the final cut was made with the same trephine bur with copious irrigation to the planned length. During the cutting, the stability of the split segment was evaluated, and the decision was made to perform the second stage of the CARS procedure. A greenstick fracture was created using a small bone carrier, and the segment was moved buccally and wedged in the surrounding buccal bone plate. The stability of the segment was then evaluated and was found to be poor. Therefore, a bone graft consisting of small particles of cancellous bovine bone (Bio-Oss, Geistlich) was moistened with normal saline and packed in the newly created intraosseous defect. The flap was then repositioned and adapted, and tension-free closure was achieved and stabilized by simple interrupted resorbable sutures (chromic gut 4/0 suture, Ethicon, Johnson & Johnson).

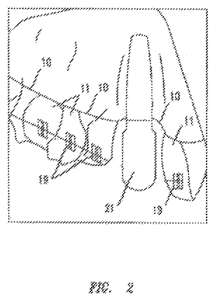

The patient returned 4 weeks later for the second surgery, and the last stage of the CARS procedure was performed under local anesthesia. A crestal incision was made at the edentulous site on the maxillary right canine with intrasulcular incisions around the buccal aspect of the right lateral incisor and the right first premolar. A full-thickness flap was then elevated without any vertical incisions. An osteotomy was made, and the implant (4.1×10 mm, BLT SLActive Roxolid, Straumann) was placed following the specific implant protocol (Fig Sa). A periapical radiograph was then taken, e.g., as in FIG. 2.

The flap was then repositioned and adapted, and tension-free closure was achieved and stabilized by interrupted resorbable 4/0 chromic gut sutures. The implant was successfully restored 9 months after implant placement.

The patient returned for follow-up every 3 months for 15 months. During this time, 2 years after implant placement, the implant and bone levels remained stable, with excellent function of the restoration.

Case 2

A 29-year-old woman presented missing a maxillary left central incisor (FIGS. 6a and 6b). The CARS technique was performed 4 weeks prior to implant placement. All procedures were performed using the same steps and materials used in Case 1, except the current patient received GBR simultaneously with implant placement The implant (4.1×10 mm, BLT SLActive Roxolid, Straumann) was placed at the central incisor site, and a GBR procedure was performed on the buccal aspect using bone graft material (Bio-Oss, particle size 1 cc, Geistlich) and a resorbable membrane (Bio-Gide, Geistlich) with tacks. Healing was uneventful.

The implant was successfully restored 12 months after placement and was followed for an additional 12 months (up to 2 years postplacement), and stable bone and soft tissue levels were seen at 24 months postplacement.

The results of the above 16 examples are set forth in Tables 1 and 2, below.

TABLE 1
Case Details of All 16 Patients with Implants Placed Using the CARS Technique

| Patient | Implant | | | Additional | Filling | Loading | Follow-up |
|---|---|---|---|---|---|---|---|
| No. | Site* | Age, y | Gender | procedure | material | time. mo | time. mo |
| 1 | 23 | 29 | F | GBR | Bio-Oss | 12 | 12 |
| 2 | 14 | 45 | M | None | Bio-Oss | 15 | 12 |
| 3 | 12, 22 | 65 | M | None | Bio-Oss | 15 | 12 |
| 4 | 21 | 22 | M | None | Bio-Oss | 13 | 12 |
| 5 | 13 | 60 | M | None | Bio-Oss | 12 | 15 |
| 6 | 11, 21 | 29 | M | GBR | Bio-Oss | 9 | 18 |
| 7 | 12 | 35 | F | None | Bio-Oss | 9 | 18 |
| 8 | 13 | 22 | F | None | Bio-Oss | 9 | 24 |
| 9 | 12 | 29 | F | None | Bio-Oss | 9 | 24 |
| 10 | 11, 21 | 34 | F | None | Bio-Oss | 9 | 18 |
| 11 | 11, 21 | 62 | F | None | Bio-Oss | 9 | 18 |
| 12 | 13 | 50 | F | None | Bio-Oss | 9 | 24 |
| 13 | 11, 21 | 51 | M | GBR | Bio-Oss | 6 | 18 |
| 14 | 24 | 65 | F | Fractured | Bio-Oss | 6 | 24 |
| 15 | 11, 21 | 50 | F | None | None | 6 | 18 |
| 16 | 12 | 30 | F | None | Bio-Oss | 6 | 18 |

CARS = Customized Alveolar Ridge-Splitting; F = female; GBR = guided bone regeneration; M = male.
All Bio-Oss (Gesitlich) filling material used small particle sizes (1 cc).
*FDI numbering system.

APPENDIX TABLE 2
Comparison Between GBR and Block
Grafts, Ridge Splitting, and CARS Techniques

| | GBR and | | |
|---|---|---|---|
| | block graft | Ridge splitting | CARS |
| Osseous | Intra- or | Intraosseous | Intraosseous |
| defect | extraosseous | | |
| Cutting | Decortication | Horizontal | Vertical cutting |
| direction | | cutting | |
| Wound size | Large | Large | Small |
| Technique | Operator- | Operator- | Operator-sensitive (easy |
| | sensitive | sensitive | learning curve with 3D |
| | | | models) |
| Incidence of use | High | High | TBD |

CARS: Customized Alveolar Ridge-Splitting,
GBR = guided bone regeneration;
TBD = to be determined.

The foregoing descriptions of embodiments of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. For example, the above embodiments describe quantitative values with respect to times and sizes of the instruments. These measurements are intended as exemplary and not limiting the invention, and do not preclude various modifications and variations within the scope of this invention.

In summary, the embodiments described hereinabove are intended to explain preferred examples of practicing the invention and to enable others skilled in the art to practice the inventions using their best experience and skills.

1. A trephine bur design drill bit comprising a first end section twist drill having a constant but small diameter cutting surface, and a second, middle section twist drill, having a rough edge surface and a gradually enlarging diameter surface extending to the second end of the trephine bur, which comprises a third section designed for connection to a drill bit driving mechanism.

2. A method for augmenting bone size of the alveolar ridge in order to improve support for a dental implant, the method comprising:
forming a full depth flap of gum tissue exposing the horizontal surface of the alveolar ridge bone;
drilling a pair of pilot holes a predetermined distance apart into the horizontal section of the alveolar ridge, the predetermined distance being determined by the size of the bone flap desired;
seating a trephine guide into the pilot holes and drilling through the guide to expand an outer portion of the bone to define the bone flap;
expanding the sides of the bone flap, causing the bone flap to bend outward to form a space between the bone flap and the inner portion of the bone;
inserting bone graft material between the outwardly bent bone flap and the opposing side of the bone; and
suturing the gum tissue flap thus securing the bone graft material in place within the outwardly bent bone flap.
